(19) United States
(12) Patent Application Publication
(10) Pub. No.: US 2016/0060193 A1
(43) Pub. Date: Mar. 3, 2016

(54) DICHLOROMETHANE PURIFICATION METHOD AND PROCESS FOR PRODUCING DIFLUOROMETHANE USING THE SAME

(71) Applicant: SHOWA DENKO K.K., Tokyo (JP)

(72) Inventors: Hiromoto OHNO, Tokyo (JP); Toshio OHI, Tokyo (JP)

(73) Assignee: SHOWA DENKO K.K., Tokyo (JP)

(21) Appl. No.: 14/787,339

(22) PCT Filed: May 8, 2014
(86) PCT No.: PCT/JP2014/062299

§ 371 Date: Oct. 27, 2015

(30) Foreign Application Priority Data
May 13, 2013 (JP) 2013-101207

(51) Int. Cl. C07C 17/389; C07C 17/383; C07C 17/20

(52) U.S. Cl. CPC C07C 17/389; C07C 17/206; C07C 17/383

(57) ABSTRACT

To provide a dichloromethane purification method which can reduce the amount of stabilizers present in dichloromethane and is feasible in industry by a simple operation, a dichloromethane purification method includes bringing dichloromethane containing at least one stabilizer selected from the group consisting of 2-methyl-2-butene, hydroquinone and resorcinol into contact in a liquid phase state with a zeolite having an average pore size of 3 to 11 Å and thereby reducing the amount of the stabilizer.

### TECHNICAL FIELD

The present invention relates to a process for producing difluoromethane by reacting dichloromethane with hydrogen fluoride in a gas phase, and to a method for purifying dichloromethane that is one of the raw materials used in the production process.

### BACKGROUND ART

Chlorofluorocarbons which seriously destroy the stratospheric ozone layer have been banned for use internationally. Further, the restrictions limit the production and use of hydrochlorofluorocarbons. On the other hand, difluoromethane (CH₂F₂) is an important compound attracting attention as an alternative refrigerant due to its being free from chlorine and its zero ozone depletion potential, low global warming potential and excellent refrigeration performance.

As known in the art, difluoromethane is conventionally produced by reacting dichloromethane with hydrogen fluoride in a gas phase in the presence of a fluorination catalyst (Patent Literature 1).

Patent Literature 2 is directed to a method for purifying raw materials for use in the production of hydrofluorocarbons. Specifically, the disclosed method is such that trichloroethylene is purified of a stabilizer that is a hydroxyl group-containing aromatic compound with use of a molecular sieve. It is described that the removal of the stabilizer can prevent a decrease in the activity of a catalyst used in the reaction of trichloroethylene and hydrogen fluoride.

Patent Literature 3 discloses an invention directed to an ethyl chloride purification method in which crude ethyl chloride containing a stabilizer and/or water is brought into contact with a zeolite and/or a carbonaceous adsorbent having an average pore size of about 3 to 11 Å in a liquid phase and thereby the amount of the stabilizer and/or water is reduced. The invention of this literature also relates to a process for producing fluoroethane using ethyl chloride purified by the method. According to the disclosed invention, a stabilizer and/or water can be removed efficiently by the simple method, and fluoroethane can be produced in an economic manner while preventing the occurrence of problems such as catalyst deterioration during the production of fluoroethane.

### CITATION LIST

### Patent Literature

Patent Literature 1: JP-A-H08-508028

Patent Literature 2: JP-A-H05-286875

Patent Literature 3: WO 2006/030656

### SUMMARY OF INVENTION

### Technical Problem

In general, various stabilizers are frequently added to raw materials used in the production of hydrofluorocarbons. For example, commercially available dichloromethane that is a raw material for difluoromethane contains several tens to several thousands of ppm of stabilizers in order for the dichloromethane to be prevented from decomposition into undesired products such as formaldehyde, hydrogen chloride and methyl chloride due to conditions such as light, water and temperature. However, when such dichloromethane is subjected to a gas-phase reaction with hydrogen fluoride in the presence of a fluorination catalyst to form the target difluoromethane, the stabilizers cause the shortening of the catalyst life or the occurrence of byproducts. Thus, the amount of such stabilizers should be reduced as much as possible.

The present invention has been made under the circumstances discussed above. It is therefore an object of the invention to provide a dichloromethane purification method which can reduce the amount of stabilizers present in dichloromethane and is feasible in industry by a simple operation. Another object of the invention is to provide a process for producing difluoromethane by reacting dichloromethane with hydrogen fluoride in the presence of a fluorination catalyst while ensuring that the catalyst deterioration and the occurrence of byproducts are inhibited and difluoromethane may be obtained in a high yield.

### Solution to Problem

The present inventors carried out extensive studies in order to achieve the above objects. As a result, the present inventors have found that the amount of stabilizers may be reduced by bringing dichloromethane containing stabilizers into contact with a zeolite in a liquid phase. The present inventors have further found that the removal or reduction of stabilizers by the above method allows the purified dichloromethane to be reacted with hydrogen fluoride in a gas phase in the presence of a fluorination catalyst so as to ensure that the catalyst deterioration and the occurrence of byproducts are inhibited and difluoromethane is obtained in a high yield. The present invention has been completed based on the findings.

Specifically, the present invention resides in, for example, the following aspects [1] to [11].

[1]

A dichloromethane purification method including bringing dichloromethane containing at least one stabilizer selected from the group consisting of 2-methyl-2-butene, hydroquinone and resorcinol into contact in a liquid phase state with a zeolite having an average pore size of 3 to 11 Å and thereby reducing the amount of the stabilizer.

[2]

The dichloromethane purification method described in [1], wherein the zeolite is at least one selected from the group consisting of molecular sieve 3A, molecular sieve 4A, molecular sieve 5A, molecular sieve 10X and molecular sieve 13X.

[3]

The dichloromethane purification method described in [1] or [2], wherein the contact between the dichloromethane and the zeolite in a liquid phase state is performed at a temperature of −15 to 65° C.

[4]

A difluoromethane production process including:

(1) a step of purifying dichloromethane by the method described in any of [1] to [3]; and

(2) a step of reacting the dichloromethane resulting from the step (1) with hydrogen fluoride in a reactor in a gas phase in the presence of a fluorination catalyst to form a gas including difluoromethane.

[5]

The difluoromethane production process described in [4], wherein the fluorination catalyst is a supported or bulk catalyst including a catalyst component containing chromium (III) oxide.

[6]

The difluoromethane production process described in [4] or [5], wherein the fluorination catalyst is a supported catalyst including a catalyst component supported on active alumina, the active alumina having a pore size distribution in which the median pore size is 50 to 400 Å and pore sizes between plus and minus 50% on the median size represent 70% or more of the distribution, the active alumina having a pore volume in the range of 0.5 to 1.6 ml/g, a purity of not less than 99.9 mass % and a sodium content of not more than 100 ppm by mass.

[7]

The difluoromethane production process described in any of [4] to [6], further including:

(3) a step of liquefying the gas including difluoromethane obtained in the step (2) and separating the resultant liquid by distillation into a high-boiling fraction and a low-boiling fraction including difluoromethane; and

(4) a step of refining difluoromethane from the low-boiling fraction obtained in the step (3).

[8]

The difluoromethane production process described in [7], wherein the step (4) includes a step of removing an acid component by bringing the difluoromethane into contact with a treatment agent including water and/or an alkaline substance.

[9]

The difluoromethane production process described in [7] or [8], wherein the step (4) includes a step of dehydrating the difluoromethane by contact with a desiccant obtained by soaking a shaped piece of molecular sieve 3A zeolite ion exchanged to exchange 20 to 60% of its sodium ions for potassium ions as expressed in an ion equivalent ratio, into an aqueous solution of sodium silicate and/or potassium silicate to attach silica to the shaped piece, recovering the shaped piece from the aqueous solution, and dehydrating and activating the shaped piece.

[10]

The difluoromethane production process described in any of [7] to [9], wherein the distillation in the step (3) is performed at a pressure in the range of 0.1 to 5 MPa.

[11]

The difluoromethane production process described in any of [7] to [10], wherein the step (3) is followed by a cycle of the step (2) and the step (3), and the step (2) following the step (3) includes supplying the high-boiling fraction to the reactor.

### Advantageous Effects of Invention

According to the dichloromethane purification method of the invention, dichloromethane may be purified by reducing or removing stabilizers present in the dichloromethane in a simple and efficient manner. According to the difluoromethane production process of the invention, difluoromethane may be produced in a high yield while preventing the catalyst deterioration and the occurrence of byproducts. The resultant difluoromethane may be used as a refrigerant or an etching gas.

### DESCRIPTION OF EMBODIMENTS

The present invention will be described in detail hereinbelow.

### Dichloromethane Purification Method

A dichloromethane purification method of the invention is characterized in that dichloromethane, more specifically, dichloromethane containing stabilizers (hereinafter, also written as “crude dichloromethane”) is brought into contact with a zeolite in a liquid phase state and thereby the amount of the stabilizers present in the dichloromethane is reduced.

Commercial dichloromethane as a raw material for the production of difluoromethane (CH₂F₂) generally contains stabilizers such as 2-methyl-2-butene, hydroquinone and resorcinol to prevent reactions such as decomposition induced by factors such as water, temperature and light, and thereby to ensure long-term stability. The amount of such stabilizers is several hundreds to several thousands of ppm by mass. Further, dichloromethane contains several tens to several hundreds of ppm by mass of water.

When such dichloromethane is subjected to a reaction with hydrogen fluoride in the presence of a fluorination catalyst to produce difluoromethane, the stabilizers give rise to adverse effects on the fluorination catalyst that is critical in the production (such as decreasing the activity or the life of the fluorination catalyst) and also cause undesired problems such as the occurrence of minor byproducts. Thus, the least amount of such stabilizers is desired. It is more desirable to eliminate the entry of such stabilizers into the step in which dichloromethane is reacted with hydrogen fluoride. Further, water present in the production of difluoromethane hydrolyzes dichloromethane to generate undesired byproducts and also causes problems such as the corrosion of reactor materials. It is therefore preferable that the amount of water be as small as possible. For example, the total amount of stabilizers and water is preferably not more than 20 ppm by mass, more preferably not more than 15 ppm by mass, and most preferably not more than 10 ppm by mass.

The average pore size of the zeolite is 3 to 11 Å, and preferably 3 to 10 Å. For example, the average pore size may be measured by a gas adsorption method using Ar gas. The above range of the average pore sizes ensures that the zeolite exhibits high adsorption performance with respect to stabilizers and water while dichloromethane is prevented from being adsorbed. In the zeolite, the silica/alumina ratio (the molar ratio of SiO₂ to Al₂O₃ constituting the zeolite) is preferably not more than 3. Preferred examples of the zeolites include molecular sieve 3A (MS-3A), molecular sieve 4A (MS-4A), molecular sieve 5A (MS-5A), molecular sieve 10X (MS-10X) and molecular sieve 13X (MS-13X).

The contact of crude dichloromethane with the zeolite in a liquid phase may be performed by a batchwise method or a continuous method.

When the dichloromethane purification method of the invention is implemented in industry, it is preferable to pass crude dichloromethane continuously through the zeolite as a fixed bed. While the liquid hourly space velocity (LHSV) of crude dichloromethane may be selected appropriately in accordance with the concentrations of stabilizers and water and the amount of crude dichloromethane to be treated, it is usually preferable that the LHSV be in the range of 1 to 80 Hr⁻¹. In implementing the dichloromethane purification method of the invention in industry, it is preferable to provide two adsorption towers which contain the zeolite as an adsorbent and are switched from one to the other to allow for continuous purification.

The contact between crude dichloromethane and the zeolite in a liquid phase is preferably performed at a temperature of −15 to 65° C., and more preferably 2 to 55° C. This range of temperatures ensures that dichloromethane may be purified while suppressing any dichloromethane decomposition reaction and without entailing special high-pressure resistant adsorption vessels (adsorption apparatuses) or additional devices such as water freeze prevention devices.

The pressure is preferably in the range of 0.05 to 1 MPa, and more preferably in the range of 0.05 to 0.6 MPa. With the pressure in this range, dichloromethane may be purified without entailing special high-pressure resistant adsorption vessels (adsorption apparatuses).

As a result of the contact of dichloromethane containing stabilizers with the zeolite in a liquid phase, the resultant dichloromethane may be purified to such an extent that the total amount of impurities (the total amount of stabilizers and water) is reduced to not more than 20 ppm by mass, more preferably not more than 15 ppm by mass, and still more preferably not more than 10 ppm by mass.

### Difluoromethane Production Process

Next, a difluoromethane production process of the invention will be described.

The difluoromethane production process of the invention is characterized in that it includes the following steps:

(1) the step of purifying dichloromethane by the inventive dichloromethane purification method described hereinabove; and

(2) the step of reacting the dichloromethane resulting from the step (1) with hydrogen fluoride in a reaction vessel in a gas phase in the presence of a fluorination catalyst to form a gas including difluoromethane.

By virtue of the step (1) being performed prior to the step (2), the fluorination catalyst used in the step (2) is prevented from deterioration (the life of the fluorination catalyst is extended), the occurrence of byproducts is suppressed, and the yield of difluoromethane is increased, thus allowing difluoromethane to be produced more efficiently and more economically. The gas including difluoromethane that is obtained in the step (2) is based on difluoromethane and sometimes contains impurities such as hydrogen chloride and chlorofluoromethane as byproducts as well as unreacted dichloromethane and hydrogen fluoride.

The fluorination catalyst is preferably a supported or bulk catalyst including a catalyst component which contains chromium (III) oxide as the main material and optionally further contains at least one element selected from the group consisting of In, Zn, Ni, Co, Mg and Al (hereinafter, the catalyst component will be also written as the catalyst component “a”). In the supported catalyst including the catalyst component “a”, the ratio of chromium (III) oxide to the whole of the supported catalyst is preferably 10 to 30 mass %.

The carrier in the fluorination catalyst is preferably active alumina which has a pore size distribution in which the median pore size is 50 to 400 Å and pore sizes between plus and minus 50% on the median size represent 70% or more of the distribution, and which has a pore volume in the range of 0.5 to 1.6 ml/g, a purity of not less than 99.9 mass % and a sodium content of not more than 100 ppm by mass.

The fluorination catalyst is more preferably a supported catalyst in which the catalyst component “a” is supported on the above active alumina (hereinafter, this catalyst will be also written as the catalyst “b”). The support ratio of the catalyst component “a” to the catalyst “b” (mass of catalyst component “a”/mass of catalyst “b”) is preferably 10 to 30 mass %.

Also preferably, at least part of the fluorination catalyst is fluorinated (namely, activated) with an agent such as hydrogen fluoride.

In the step (2), the reaction temperature is preferably in the range of 170° C. to 350° C., and more preferably 200 to 330° C. This range of the reaction temperatures ensures that the occurrence of byproducts and the deterioration of the catalyst are suppressed and that difluoromethane is produced with a high reaction yield.

The molar ratio of the reaction raw materials, namely, hydrogen fluoride to dichloromethane (HF/CH₂Cl₂) is preferably in the range of 3 to 30, and more preferably in the range of 5 to 20. When the molar ratio is in this range, the occurrence of byproducts may be suppressed and difluoromethane may be produced with high selectivity and high economic efficiency.

The ratio of the volume velocity (F) at which the reaction raw materials are supplied, to the volume (V) of the catalyst in the reactor, namely, the space velocity (SV) is 50 to 100000 h⁻¹.

The pressure during the reaction is preferably in the range of 0.1 to 1.0 MPa, and more preferably 0.1 to 0.7 MPa. With the pressure in this range, difluoromethane may be produced by a simple operation economically without entailing any special high-pressure resistant reaction vessel (reaction apparatus).

The difluoromethane production process of the invention may further include:

(3) a step of liquefying the gas including difluoromethane obtained in the step (2) and separating the resultant liquid by distillation into a high-boiling fraction and a low-boiling fraction including difluoromethane; and

(4) a step of refining difluoromethane from the low-boiling fraction obtained in the step (3).

The addition of the step (3) and the step (4) to the difluoromethane production process of the invention makes it possible to obtain difluoromethane having a higher purity.

In the case where the distillation in the step (3) is performed in a distillation column, the gas including difluoromethane obtained in the step (2) may be introduced into the distillation column by, for example, cooling the gas and pumping the resultant liquid into the distillation column, or by transporting the gas into the distillation column with a compressor. In view of factors such as facility costs and operability, a preferred method is to cool the gas and pump the resultant liquid into the distillation column. From the points of view of economic efficiency and operability, the pressure in operating the distillation column is preferably 0.1 to 5 MPa, and more preferably 0.3 to 3 MPa.

The difluoromethane production process of the invention may involve two distillation columns. First, in the step (3), the gas including difluoromethane obtained in the step (2), specifically, the gas including difluoromethane as the main component and also containing impurities may be liquefied by cooling and thereafter introduced into the first distillation column, and a fraction (a top fraction) which is based on the target difluoromethane and contains hydrogen chloride as a byproduct may be discharged from the top of the distillation column. In the step (4), the top fraction may be introduced into the second distillation column to discharge hydrogen chloride from the top of the column and to recover the target difluoromethane through the bottom of the column.

The difluoromethane resulting from the step (3), specifically, the difluoromethane in the low-boiling fraction or, in the case where two distillation columns are used, the difluoromethane as the bottom fraction in the second distillation column contains a trace amount of acids (such as HF and HCl). It is therefore preferable to bring the difluoromethane into contact with a treatment agent including water and/or an alkali to remove such acids. Examples of the alkali-containing treatment agents include aqueous alkali solutions and alkali-containing solid materials (for example, soda lime). The acid concentration in the difluoromethane after the acid removal treatment is preferably not more than 1.0 ppm by mass (measurement: ion chromatography).

In the case where the deacidified difluoromethane contains water, the process preferably involves a step of dehydrating (drying) the difluoromethane.

If molecular sieve 3A, molecular sieve 4A or molecular sieve 5A is used as the dehydrating agent in this dehydration (drying) step, the difluoromethane is adsorbed to the dehydrating agent because of its small molecular size and is decomposed due to factors such as adsorption heat. To avoid this problem, the dehydrating agent (desiccant) is preferably a dehydrating agent (desiccant) obtained by soaking a shaped piece of molecular sieve 3A zeolite ion exchanged to exchange 20 to 60% of its sodium ions for potassium ions as expressed in an ion equivalent ratio, into an aqueous solution of sodium silicate and/or potassium silicate to attach silica to the shaped piece, recovering the shaped piece from the aqueous solution, and dehydrating and activating the shaped piece. The water concentration in the difluoromethane dehydrated (dried) with this dehydrating agent (desiccant) is preferably not more than 10 ppm by mass (measurement: Karl Fischer's method).

The high-boiling fraction obtained in the step (3) includes components such as dichloromethane and hydrogen fluoride used as the raw materials for difluoromethane, and chlorofluoromethane as an intermediate. In the difluoromethane production process of the invention, the step (3) may be followed by a cycle of the step (2) and the step (3), and the step (2) following the step (3) may include supplying the high-boiling fraction to the reactor.

The supply of the high-boiling fraction to the reactor may take place directly with respect to the whole of the high-boiling fraction or selectively with respect to a specific component present in the high-boiling fraction.

### EXAMPLES

The present invention will be described in greater detail based on examples hereinbelow without limiting the scope of the invention to such examples.

### Raw Material Example 1

Commercial dichloromethane (containing a stabilizer) was analyzed by gas chromatography and the content of 2-methyl-2-butene (trivial name: isoamylene) in dichloromethane was determined to be 318 ppm by mass. P-hydroquinone and water were absent.

### Raw Material Example 2

Commercial dichloromethane (containing a stabilizer) was analyzed by gas chromatography to determine the content of p-hydroquinone and by a Karl Fischer's method to quantify water. The measurements showed that the dichloromethane contained 272 ppm by mass of p-hydroquinone and 129 ppm by mass of water. 2-Methyl-2-butene was absent.

### Dichloromethane Purification Method

### Example 1

A 100 ml volume stainless steel vessel was loaded with 20 g of molecular sieve 5A (manufactured by UNION SHOWA K.K.: average pore size: 4.2 Å, silica/alumina ratio=2.0). Vacuum drying was performed. Thereafter, 80 g of dichloromethane of Raw Material Example 1 was added while cooling the vessel. While keeping the temperature at ambient (approximately 23° C.), the mixture was stirred intermittently. After approximately 7 hours from the addition of dichloromethane, a portion of the liquid phase was sampled and was analyzed by gas chromatography. As a result, the content of 2-methyl-2-butene in dichloromethane had been reduced to 1 ppm by mass (detection lower limit: 0.5 ppm by mass).

### Example 2

A 100 ml volume stainless steel vessel was loaded with 30 g of molecular sieve 5A (manufactured by UNION SHOWA K.K.: average pore size: 4.2 Å, silica/alumina ratio=2.0). Vacuum drying was performed. Thereafter, 70 g of dichloromethane of Raw Material Example 2 was added while cooling the vessel. While keeping the temperature at ambient (approximately 25° C.), the mixture was stirred intermittently. After approximately 7 hours from the addition of dichloromethane, a portion of the liquid phase was sampled and was analyzed by gas chromatography and a Karl Fischer's method. As a result, the content of p-hydroquinone had been reduced to 1 ppm by mass (detection lower limit: 0.5 ppm by mass) and the content of water had been reduced to 4 ppm by mass (detection lower limit: 0.5 ppm by mass).

### Example 3

A 200 ml volume stainless steel vessel was loaded with 30 g of molecular sieve 13X (manufactured by UNION SHOWA K.K.: average pore size: 10 Å, silica/alumina ratio=2.5) and 15 g of molecular sieve 3A (manufactured by UNION SHOWA K.K.: average pore size: 3 Å, silica/alumina ratio=2.0). Vacuum drying was performed. Thereafter, 120 g of dichloromethane of Raw Material Example 2 was added while cooling the vessel. While keeping the temperature at 10° C., the mixture was stirred intermittently. After approximately 7 hours from the addition of dichloromethane, a portion of the liquid phase was sampled and was analyzed by gas chromatography and a Karl Fischer's method. As a result, the content of p-hydroquinone had been reduced to 1 ppm by mass and the content of water had been reduced to 3 ppm by mass.

### Difluoromethane Production Process

### Catalyst Preparation Example 1

As a fluorination catalyst carrier, active alumina (Nikki-Universal Co., Ltd., trade name: NST-7) was used which had a pore size distribution in which the median pore size was 50 to 400 Å and pore sizes between plus and minus 50% on the median size represented 70% or more of the distribution, and which had a pore volume in the range of 0.5 to 1.6 ml/g, a purity of not less than 99.9 mass % and a sodium content of not more than 100 ppm by mass.

Chromium chloride (CrCl₃.6H₂O) weighing 191.5 g was added to 132 ml of pure water. The mixture was heated to 70 to 80° C. on a hot water bath to give a solution, which was cooled to room temperature. Thereafter, 400 g of the active alumina was soaked in the solution and was caused to absorb the whole amount of the solution. Next, the wet active alumina was dried to a solid state on a hot water bath at 90° C. and was further dried in a hot air circulation dryer for 3 hours. The resultant dried product was packed into an INCONEL reactor and was fluorinated (activated) by being subjected to a nitrogen-diluted hydrogen fluoride flow and then to a 100% hydrogen fluoride flow at normal pressure and 330° C. In the manner described above, a fluorination catalyst 1 was obtained.

### Catalyst Preparation Example 2

A fluorination catalyst 2 was obtained in the same manner as in Catalyst Preparation Example 1, except that the solution of 191.5 g of chromium chloride was replaced by a solution of 191.5 g of chromium chloride and 16.57 g of zinc chloride (ZnCl₂).

### Examples 4 and 5 and Comparative Example 1

### Example 4

An INCONEL 600 reactor 2.54 cm in inner diameter and 1 m in length was loaded with 80 ml of the catalyst prepared in Catalyst Preparation Example 1 (the fluorination catalyst 1). While passing nitrogen gas, the temperature and the pressure in the reactor were maintained at 250° C. and 0.3 MPa. Subsequently, hydrogen fluoride was supplied to the reactor at 72.85 NL/hr and the supply of nitrogen gas was terminated. Thereafter, dichloromethane obtained in the same manner as in Example 1 except that the operation was scaled up was evaporated and supplied at 6.10 NL/hr, thereby initiating the reaction between dichloromethane and hydrogen fluoride. After approximately 8 hours from the initiation of the reaction, the gas discharged from the outlet of the reactor was brought into contact with an aqueous alkali solution to remove acids and was analyzed by gas chromatography. The results are described below.

| | | | |
|---|---|---|---|
| CH₂F₂ | 89.9557 | CH₂ClF | 8.1014 |
| CH₂Cl₂ | 1.9281 | Others | 0.0148 |

(Unit: vol %)

Thereafter, the reaction was continuously performed. After approximately 48 hours from the initiation of the reaction, the gas discharged from the reactor outlet was brought into contact with an aqueous alkali solution to remove acids and was analyzed by gas chromatography. The results are described below.

| | | | |
|---|---|---|---|
| CH₂F₂ | 90.9925 | CH₂ClF | 8.0037 |
| CH₂Cl₂ | 0.9886 | Others | 0.0152 |

(Unit: vol %)

### Example 5

Next, the dichloromethane was changed to dichloromethane obtained in the same manner as in Example 2 except that the operation was scaled up, and the reaction was continuously performed under the same reaction conditions as described above. After approximately 24 hours after the dichloromethane was changed, the gas discharged from the reactor outlet was brought into contact with an aqueous alkali solution to remove acids and was analyzed by gas chromatography. The results are described below.

| | | | |
|---|---|---|---|
| CH₂F₂ | 91.3526 | CH₂ClF | 7.9205 |
| CH₂Cl₂ | 0.7119 | Others | 0.0150 |

(Unit: vol %)

### Comparative Example 1

Next, the dichloromethane (purified) was changed to dichloromethane of Raw Material Example 1 (unpurified), and the reaction was continuously performed under the same reaction conditions as described above. After approximately 48 hours after the dichloromethane was changed, the gas discharged from the reactor outlet was cleaned of acids with an aqueous alkali solution and was analyzed by gas chromatography. The results are described below.

| | | | |
|---|---|---|---|
| CH₂F₂ | 86.3325 | CH₂ClF | 8.5829 |
| CH₂Cl₂ | 5.0404 | Others | 0.0442 |

(Unit: vol %)

As clear from the results, the use of unpurified dichloromethane resulted in a decrease in the yield of the target difluoromethane and an increase in the amount of impurities (Others).

### Example 6 and Comparative Example 2

### Example 6

An INCONEL 600 reactor 2.54 cm in inner diameter and 1 m in length was loaded with 80 ml of the catalyst prepared in Catalyst Preparation Example 2 (the fluorination catalyst 2). The temperature was increased while passing nitrogen gas, and the temperature and the pressure were maintained at 250° C. and 0.3 MPa. Subsequently, hydrogen fluoride was supplied at 73.85 NL/hr and the supply of nitrogen gas was terminated. Thereafter, dichloromethane obtained in the same manner as in Example 1 except that the operation was scaled up was supplied at 6.10 NL/hr, thereby initiating the reaction. After approximately 8 hours from the initiation of the reaction, the gas discharged from the outlet of the reactor was brought into contact with an aqueous alkali solution to remove acids and was analyzed by gas chromatography. The results are described below.

| | | | |
|---|---|---|---|
| CH₂F₂ | 89.1226 | CH₂ClF | 8.4255 |
| CH₂Cl₂ | 2.4365 | Others | 0.0154 |

(Unit: vol %)

### Comparative Example 2

Subsequently, the dichloromethane (purified) was changed to dichloromethane of Raw Material Example 2 (unpurified), and the reaction was continuously performed under the same reaction conditions as described above. After approximately 45 hours after the dichloromethane was changed, the gas discharged from the reactor outlet was brought into contact with an aqueous alkali solution to remove acids and was analyzed by gas chromatography. The results are described below.

| | | | |
|---|---|---|---|
| CH₂F₂ | 85.8890 | CH₂ClF | 11.0225 |
| CH₂Cl₂ | 3.0446 | Others | 0.0439 |

(Unit: vol %)

As clear from the results, the use of unpurified dichloromethane resulted in a decrease in the yield of the target difluoromethane and an increase in the amount of impurities (Others).

### Example 7

The same operations as in Examples 4 and 5 were performed. The gas discharged from the reactor outlet was recovered into a vessel equipped with a cooling device, and was liquefied by being cooled. The recovered liquid was introduced into a distillation apparatus and was distilled at a pressure of 0.65 MPa. The distillation apparatus was a distillation column equipped with a condenser which had 20 theoretical plates (36 actual plates). Mainly hydrogen chloride and difluoromethane were recovered through the top of the distillation column, while high-boiling components based on hydrogen fluoride, chlorofluoromethane and dichloromethane were recovered through the bottom of the column. The hydrogen chloride and difluoromethane recovered from the column top were brought into contact with a 2% aqueous potassium hydroxide solution at a temperature of about 5° C., and thereby acids were removed. The concentration of acids (HF+HCl) in the difluoromethane was analyzed by ion chromatography. The acid concentration was measured to be 0.7 ppm by mass.

Next, the difluoromethane was further brought into contact with an aqueous alkali solution and then with a column filled with a desiccant. Thereafter, the difluoromethane was subjected to an analysis by a Karl Fischer's method, and the water content was measured to be 5 ppm by mass. The desiccant was one which had been obtained by a process in which 100 g of 3A zeolite beads with a zeolite content of 80% having a potassium ion exchange ratio of 33% and an average particle size of 2.1 mm in diameter were thoroughly soaked into an aqueous solution obtained by adding 20 parts by mass of water to 100 parts by mass of a 40 mass % aqueous sodium silicate solution, and the beads were held as such at room temperature for 24 hours, recovered, washed with water, dried at 200° C. for 2 hours, and activated by being heated at 450° C. for 2 hours.

### INDUSTRIAL APPLICABILITY

According to the present invention, difluoromethane that has important applications in industry such as use as a refrigerant may be obtained with high purity.

1. A dichloromethane purification method comprising bringing dichloromethane containing at least one stabilizer selected from the group consisting of 2-methyl-2-butene, hydroquinone and resorcinol into contact in a liquid phase state with a zeolite having an average pore size of 3 to 11 Å and thereby reducing the amount of the stabilizer.

2. The dichloromethane purification method according to claim 1, wherein the zeolite is at least one selected from the group consisting of molecular sieve 3A, molecular sieve 4A, molecular sieve 5A, molecular sieve 10X and molecular sieve 13X.

3. The dichloromethane purification method according to claim 1, wherein the contact between the dichloromethane and the zeolite in a liquid phase state is performed at a temperature of −15 to 65° C.

4. A difluoromethane production process comprising:
(1) a step of purifying dichloromethane by the method described in claim 1; and
(2) a step of reacting the dichloromethane resulting from the step (1) with hydrogen fluoride in a reactor in a gas phase in the presence of a fluorination catalyst to form a gas including difluoromethane.

5. The difluoromethane production process according to claim 4, wherein the fluorination catalyst is a supported or bulk catalyst including a catalyst component containing chromium (III) oxide.

6. The difluoromethane production process according to claim 4, wherein the fluorination catalyst is a supported catalyst including a catalyst component supported on active alumina, the active alumina having a pore size distribution in which the median pore size is 50 to 400 Å and pore sizes between plus and minus 50% on the median size represent 70% or more of the distribution, the active alumina having a pore volume in the range of 0.5 to 1.6 ml/g, a purity of not less than 99.9 mass % and a sodium content of not more than 100 ppm by mass.

7. The difluoromethane production process according to claim 4, further comprising:
(3) a step of liquefying the gas including difluoromethane obtained in the step (2) and separating the resultant liquid by distillation into a high-boiling fraction and a low-boiling fraction including difluoromethane; and
(4) a step of refining difluoromethane from the low-boiling fraction obtained in the step (3).

8. The difluoromethane production process according to claim 7, wherein the step (4) includes a step of removing an acid component by bringing the difluoromethane into contact with a treatment agent including water and/or an alkaline substance.

9. The difluoromethane production process according to claim 7, wherein the step (4) includes a step of dehydrating the difluoromethane by contact with a desiccant obtained by soaking a shaped piece of molecular sieve 3A zeolite ion exchanged to exchange 20 to 60% of its sodium ions for potassium ions as expressed in an ion equivalent ratio, into an aqueous solution of sodium silicate and/or potassium silicate to attach silica to the shaped piece, recovering the shaped piece from the aqueous solution, and dehydrating and activating the shaped piece.

10. The difluoromethane production process according to claim 7, wherein the distillation in the step (3) is performed at a pressure in the range of 0.1 to 5 MPa.

11. The difluoromethane production process according to claim 7, wherein the step (3) is followed by a cycle of the step (2) and the step (3), and the step (2) following the step (3) includes supplying the high-boiling fraction to the reactor.

12. The dichloromethane purification method according to claim 2, wherein the contact between the dichloromethane and the zeolite in a liquid phase state is performed at a temperature of −15 to 65° C.
